(19) United States
(12) Patent Application Publication
(10) Pub. No.: US 2005/0123904 A1
(43) Pub. Date: Jun. 9, 2005

(54) Methods and compositions for modulating herpesviral replication and transcription activator

(75) Inventors: Josephine Harada, San Diego, CA (US); Christian Nelson, San Diego, CA (US); Sumit Chanda, La Jolla, CA (US)

Correspondence Address: GENOMICS INSTITUTE OF THE;NOVARTIS RESEARCH FOUNDATION, 10675 JOHN JAY HOPKINS DRIVE, SUITE E225, SAN DIEGO, CA, 92121-1127, US

(73) Assignee: IRM LLC, Hamilton (BM)

(21) Appl. No.: 10/920,595

(22) Filed: Aug. 17, 2004

Related U.S. Application Data
(60) Provisional application No. 60/496,047, filed on Aug. 18, 2003.

(51) Int. Cl. C12Q 1/70; A61K 38/16; A61K 48/00

(52) U.S. Cl. 435/5; 514/12; 514/44

(57) ABSTRACT

This invention provides novel polypeptides that modulate activities of the replication and transcription activator (RTA) of Kaposi's sarcoma-associated herpesvirus (KSHV). The invention also provides methods for screening modulators of RTA activities. The methods comprise first screening test agents for modulators of an RTA-modulatory polypeptide and then further screening the identified modulating agents for modulators of RTA activities. The invention further provides methods and pharmaceutical compositions for treating diseases and conditions associated with infections of gammaherpesviruses.

### CROSS-REFERENCE TO RELATED APPLICATIONS

This application claims the benefit of priority under 35 U.S.C. §119(e) to U.S. Provisional Patent Application No. 60/496,047, filed Aug. 18, 2003. The disclosure of the priority application is incorporated herein by reference in its entirety and for all purposes.

### FIELD OF THE INVENTION

The present invention generally relates to methods for identifying modulators of herpesviral replication and transcription activator (RTA) and therapeutic applications of such modulators. More particularly, the invention pertains to novel RTA modulators that regulate RTA activities, and to methods of using such modulators to modulate RTA activities in vivo.

### BACKGROUND OF THE INVENTION

Kaposi's sarcoma-associated herpesvirus (KSHV), also known as human herpesvirus 8, is an etiological agent of Kaposi's sarcoma (KS). Clinical forms of KS include AIDS-associated KS, classic KS, endemic forms of KS, and renal transplant-related KS. KSHV is also associated with primary effusion lymphoma (PEL) and multicentric Castleman's disease, two AIDS-related lymphoproliferative diseases. KSHV belongs to the gammaherpesvirus subfamily of herpesviridae. Members of this subfamily also include the Epstein-Barr virus.

The replication and transcription activator, RTA, also referred to as ORF50, Lyta, and ART, is an immediate-early gene product of KSHV. RTA, especially its DNA-binding domain, is well conserved among all gammaherpesviruses, e.g., Epstein-Barr virus (Manet et al., EMBO J. 8:1819-1826, 1989); bovine herpesvirus 4 (van Santen et al., J. Virol. 67:773-784, 1993); herpesvirus saimiri (Whitehouse et al., J. Virol. 71:2550-2554, 1997); and murine gammaherpesvirus 68 (Wu et al., J. Virol. 74:3659-3667, 2000). It has been shown to play a central role in the switch of the viral life cycle from latency to lytic replication. Indeed, the expression of KSHV RTA alone was shown to be necessary and sufficient for the initiation of the full lytic replication. KSHV RTA was shown to transactivate the expression of several downstream genes. In addition, RTA has been shown to autostimulate its own expression (Deng et al., J. Gen. Virol. 81: 3043-3048, 2000; Gradoville et al., J. Virol. 74:6207-6212, 2000; Ragoczy et al., J. Virol. 75:5240-5251, 2001; and Wu et al., J. Virol. 74:3659-3667, 2000).

Given the pivotal role of RTA in gammaherpesviral life cycle, modulation of RTA activities (e.g., inhibition) would provide means to interrupt viral lytic replication of gammaherpesviruses (e.g., KSHV and Epstein-Barr virus) and hence progression of viral infection. There is a need in the art for novel methods and compositions for treating diseases and conditions associated with infections of herpesviruses. The instant invention fulfills this and other needs.

### SUMMARY OF THE INVENTION

The present invention relates to novel RTA-modulatory polypeptides, methods for screening for modulators of RTA, and methods for treating diseases and conditions associated with infections of gammaherpesviruses (e.g., KSHV and Epstein-Barr virus).

In one aspect, the invention provides methods for identifying agents that modulate the replication and transcription activator (RTA) of a gammaherpesvirus. The methods entail (a) assaying a biological activity of an RTA-modulatory polypeptide encoded by a polynucleotide selected from the members listed in Table 1, or a fragment of said polypeptide, in the presence of a test agent to identify one or more modulating agents that modulate the biological activity of the polypeptide; and (b) testing one or more of the modulating agents for ability to modulate a biological activity of the RTA.

In some of the methods, the gammaherpesvirus is human Kaposi's sarcoma virus (human herpesvirus 8) or Epstein-Barr virus. In some methods, the modulating agents inhibit the biological activity of the polypeptide. In some methods, (b) comprises testing the modulating agents for ability to modulate RTA in regulating expression of an RTA responsive gene. In some other methods, (b) comprises testing the modulating agents for ability to modulate RTA in inducing expression of a second polynucleotide that is operably linked to an RTA response element. For example, the second polynucleotide can encode a reporter polypeptide.

In some of the methods, the testing for ability to modulate the biological activity of the RTA comprises providing a cell or cell lysate that comprises the second polynucleotide that is operably linked to the RTA response element; contacting the cell or cell lysate with a test agent; and detecting an increase or decrease in expression of the second polynucleotide in the presence of the test agent compared to expression of the second polynucleotide in the absence of the test agent.

In some methods, (b) comprises testing the modulating agents for ability to modulate expression level of the RTA. In some methods, the RTA-modulatory polypeptide is a kinase. In some methods, the assaying of the biological activity of the RTA-modulatory polypeptide occurs in a cell. In some of these methods, the RTA-modulatory polypeptide is expressed from said polynucleotide that has been introduced into the cell.

In another aspect, the invention provides methods for identifying an agent that modulates expression of an RTA responsive gene. The methods incur (a) contacting a test agent with an RTA-modulatory polypeptide encoded by a polynucleotide selected from the members listed in Table 1; (b) detecting a change in an activity of said RTA-modulatory polypeptide relative to the activity in the absence of the test agent; and (c) detecting a change of expression level of the RTA responsive gene in the presence of the test agent identified in (b) relative to expression level of the RTA responsive gene in the absence of the test agent. In some of these methods, (a) and (b) are performed in a cell.

In another aspect, methods for inhibiting replication of a gammaherpesvirus in a subject are provided. These methods entail administering to the subject a pharmaceutical composition comprising an effective amount of a modulator of the replication and transcription activator (RTA) of the gammaherpesvirus. The RTA modulator to be employed in these methods is identified by (a) assaying a biological activity of an RTA-modulatory polypeptide encoded by a polynucleotide selected from the members listed in Table 1, or a fragment of said polypeptide, in the presence of a test agent to identify one or more modulating agents that inhibit the biological activity of the polypeptide; and (b) testing one or more of the modulating agents for ability to inhibit transcription-regulating activity of the RTA. Some of these methods are specifically directed to inhibiting replication of human Kaposi's sarcoma virus (human herpesvirus 8) or Epstein-Barr virus. Typically, these methods are directed to human subjects.

A further understanding of the nature and advantages of the present invention may be realized by reference to the remaining portions of the specification and claims.

### DETAILED DESCRIPTION

The present invention provides methods for identifying modulators of the replication and transcription activator (RTA) of gammaherpesviruses, e.g., KSHV or Epstein-Barr virus. The invention also provides methods for modulating RTA activities in vivo and for treating diseases or conditions associated with infections of gammaherpesviruses. The following sections provide guidance for making and using the compositions of the invention, and for carrying out the methods of the invention.

I. Definitions

Unless defined otherwise, all technical and scientific terms used herein have the same meaning as commonly understood by those of ordinary skill in the art to which this invention pertains. The following references provide one of skill with a general definition of many of the terms used in this invention: Singleton et al., DICTIONARY OF MICROBIOLOGY AND MOLECULAR BIOLOGY (2d ed. 1994); THE CAMBRIDGE DICTIONARY OF SCIENCE AND TECHNOLOGY (Walker ed., 1988); and Hale & Marham, THE HARPER COLLINS DICTIONARY OF BIOLOGY (1991). In addition, the following definitions are provided to assist the reader in the practice of the invention.

The term “agent” or “test agent” includes any substance, molecule, element, compound, entity, or a combination thereof. It includes, but is not limited to, e.g., protein, polypeptide, small organic molecule, polysaccharide, polynucleotide, and the like. It can be a natural product, a synthetic compound, or a chemical compound, or a combination of two or more substances. Unless otherwise specified, the terms “agent”, “substance”, and “compound” can be used interchangeably.

The term “analog” is used herein to refer to a molecule that structurally resembles a reference molecule but which has been modified in a targeted and controlled manner, by replacing a specific substituent of the reference molecule with an alternate substituent. Compared to the reference molecule, an analog would be expected, by one skilled in the art, to exhibit the same, similar, or improved utility. Synthesis and screening of analogs, to identify variants of known compounds having improved traits (such as higher binding affinity for a target molecule) is an approach that is well known in pharmaceutical chemistry.

As used herein, “contacting” has its normal meaning and refers to combining two or more molecules (e.g., a test agent and a polypeptide) or combining molecules and cells (e.g., a test agent and a cell). Contacting can occur in vitro, e.g., combining two or more agents or combining a test agent and a cell or a cell lysate in a test tube or other container. Contacting can also occur in a cell or in situ, e.g., contacting two polypeptides in a cell by coexpression in the cell of recombinant polynucleotides encoding the two polypeptides, or in a cell lysate.

A “heterologous sequence” or a “heterologous nucleic acid,” as used herein, is one that originates from a source foreign to the particular host cell, or, if from the same source, is modified from its original form. Thus, a heterologous gene in a host cell includes a gene that, although being endogenous to the particular host cell, has been modified. Modification of the heterologous sequence can occur, e.g., by treating the DNA with a restriction enzyme to generate a DNA fragment that is capable of being operably linked to the promoter. Techniques such as site-directed mutagenesis are also useful for modifying a heterologous nucleic acid.

The term “homologous” when referring to proteins and/or protein sequences indicates that they are derived, naturally or artificially, from a common ancestral protein or protein sequence. Similarly, nucleic acids and/or nucleic acid sequences are homologous when they are derived, naturally or artificially, from a common ancestral nucleic acid or nucleic acid sequence. Homology is generally inferred from sequence similarity between two or more nucleic acids or proteins (or sequences thereof). The precise percentage of similarity between sequences that is useful in establishing homology varies with the nucleic acid and protein at issue, but as little as 25% sequence similarity is routinely used to establish homology. Higher levels of sequence similarity, e.g., 30%, 40%, 50%, 60%, 70%, 80%, 90%, 95% or 99% or more can also be used to establish homology.

A “host cell,” as used herein, refers to a prokaryotic or eukaryotic cell that contains heterologous DNA that has been introduced into the cell by any means, e.g., transfection, electroporation, calcium phosphate precipitation, microinjection, transformation, viral infection, and/or the like.

The term “sequence identity” in the context of two nucleic acid sequences or amino acid sequences refers to the residues in the two sequences which are the same when aligned for maximum correspondence over a specified comparison window. A “comparison window” refers to a segment of at least about 20 contiguous positions, usually about 50 to about 200, more usually about 100 to about 150 in which a sequence may be compared to a reference sequence of the same number of contiguous positions after the two sequences are aligned optimally. Methods of alignment of sequences for comparison are well-known in the art. Optimal alignment of sequences for comparison may be conducted by the local homology algorithm of Smith and Waterman (1981) Adv. Appl. Math. 2:482; by the alignment algorithm of Needleman and Wunsch (1970) J. Mol. Biol. 48:443; by the search for similarity method of Pearson and Lipman (1988) Proc. Nat. Acad. Sci U.S.A. 85:2444; by computerized implementations of these algorithms (including, but not limited to CLUSTAL in the PC/Gene program by Intelligentics, Mountain View, Calif.; and GAP, BESTFIT, BLAST, FASTA, or TFASTA in the Wisconsin Genetics Software Package, Genetics Computer Group (GCG), 575 Science Dr., Madison, Wis., U.S.A.). The CLUSTAL program is well described by Higgins and Sharp (1988) Gene 73:237-244; Higgins and Sharp (1989) CABIOS 5:151-153; Corpet et al. (1988) Nucleic Acids Res. 16:10881-10890; Huang et al (1992) Computer Applications in the Biosciences 8:155-165; and Pearson et al. (1994) Methods in Molecular Biology 24:307-331. Alignment is also often performed by inspection and manual alignment. In one class of embodiments, the polypeptides herein are at least 70%, generally at least 75%, optionally at least 80%, 85%, 90%, 95% or 99% or more identical to a reference polypeptide, e.g., an RTA-modulatory polypeptide encoded by a polynucleotide in Table 1, e.g., as measured by BLASTP (or CLUSTAL, or any other available alignment software) using default parameters. Similarly, nucleic acids can also be described with reference to a starting nucleic acid, e.g., they can be 50%, 60%, 70%, 75%, 80%, 85%, 90%, 95%, 99% or more identical to a reference nucleic acid, e.g., a polynucleotide in Table 1, e.g., as measured by BLASTN (or CLUSTAL, or any other available alignment software) using default parameters.

A “substantially identical” nucleic acid or amino acid sequence refers to a nucleic acid or amino acid sequence which comprises a sequence that has at least 90% sequence identity to a reference sequence using the programs described above (preferably BLAST) using standard parameters. The sequence identity is preferably at least 95%, more preferably at least 98%, and most preferably at least 99%. For example, the BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89:10915 (1989)). Percentage of sequence identity is determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Preferably, the substantial identity exists over a region of the sequences that is at least about 50 residues in length, more preferably over a region of at least about 100 residues, and most preferably the sequences are substantially identical over at least about 150 residues. In a most preferred embodiment, the sequences are substantially identical over the entire length of the coding regions.

The term “modulate” with respect to a biological activity of a reference protein or its fragment refers to a change in the expression level or other biological activities of the protein. For example, modulation may cause an increase or a decrease in expression level of the reference protein, enzymatic modification (e.g., phosphorylation) of the protein, binding characteristics (e.g., binding to a target polynucleotide), or any other biological, functional, or immunological properties of the reference protein. The change in activity can arise from, for example, an increase or decrease in expression of one or more genes that encode the reference protein, the stability of an mRNA that encodes the protein, translation efficiency, or from a change in other biological activities of the reference protein. The change can also be due to the activity of another molecule that modulates the reference protein (e.g., a kinase which phosphorylates the reference protein).

Modulation of a reference protein can be up-regulation (i.e., activation or stimulation) or down-regulation (i.e. inhibition or suppression). The mode of action of a modulator of the reference protein can be direct, e.g., through binding to the protein or to genes encoding the protein, or indirect, e.g., through binding to and/or modifying (e.g., enzymatically) another molecule which otherwise modulates the reference protein.

The term “operably linked” refers to a functional relationship between two or more polynucleotide (e.g., DNA) segments. Typically, it refers to the functional relationship of a transcriptional regulatory sequence to a transcribed sequence. For example, a transcription regulatory sequence is operably linked to a coding sequence if it modulates the transcription of the coding sequence in an appropriate host cell or other expression system. Generally, transcriptional regulatory sequences that are operably linked to a transcribed sequence are physically contiguous to the transcribed sequence, i.e., they are cis-acting. However, some transcription regulatory sequences, such as enhancers or response elements, need not be physically contiguous or located in close proximity to the coding sequences whose transcription they enhance. A polylinker provides a convenient location for inserting coding sequences so the genes are operably linked to a transcription regulatory sequence. Polylinkers are polynucleotide sequences that comprise a series of three or more closely spaced restriction endonuclease recognition sequences.

The term “polypeptide” is used interchangeably herein with the terms “polypeptides” and “protein(s)”, and refers to a polymer of amino acid residues, e.g., as typically found in proteins in nature. A “mature protein” is a protein which is full-length and which, optionally, includes glycosylation or other modifications typical for the protein in a given cell membrane.

The promoter region of a gene includes the transcription regulatory elements that typically lie 5′ to a structural gene. If a gene is to be activated, proteins known as transcription factors attach to the promoter region of the gene. This assembly resembles an “on switch” by enabling an enzyme to transcribe a second genetic segment from DNA into RNA. In most cases the resulting RNA molecule serves as a template for synthesis of a specific protein; sometimes RNA itself is the final product. The promoter region may be a normal cellular promoter or an oncopromoter.

The term “recombinant” has the usual meaning in the art, and refers to a polynucleotide synthesized or otherwise manipulated in vitro (e.g., “recombinant polynucleotide”), to methods of using recombinant polynucleotides to produce gene products in cells or other biological systems, or to a polypeptide (“recombinant protein”) encoded by a recombinant polynucleotide. When used with reference to a cell, the term indicates that the cell replicates a heterologous nucleic acid, or expresses a peptide or protein encoded by a heterologous nucleic acid. Recombinant cells can contain genes that are not found within the native (non-recombinant) form of the cell. Recombinant cells can also contain genes found in the native form of the cell wherein the genes are modified and re-introduced into the cell by artificial means. The term also encompasses cells that contain a nucleic acid endogenous to the cell that has been modified without removing the nucleic acid from the cell; such modifications include those obtained by gene replacement, site-specific mutation, and related techniques.

A “recombinant expression vector” or simply an “expression vector” is a nucleic acid construct, generated recombinantly or synthetically, that has control elements that are capable of affecting expression of a structural gene that is operably linked to the control elements in hosts compatible with such sequences. Expression vectors include at least promoters and optionally, transcription termination signals. Typically, the recombinant expression vector includes at least a nucleic acid to be transcribed and a promoter. Additional factors necessary or helpful in effecting expression can also be used as described herein. For example, transcription termination signals, enhancers, and other nucleic acid sequences that influence gene expression, can also be included in an expression vector.

Transcription refers to the process involving the interaction of an RNA polymerase with a gene, which directs the expression as RNA of the structural information present in the coding sequences of the gene. The process includes, but is not limited to the following steps: (1) transcription initiation, (2) transcript elongation, (3) transcript splicing, (4) transcript capping, (5) transcript termination, (6) transcript polyadenylation, (7) nuclear export of the transcript, (8) transcript editing, and (9) stabilization of the transcript.

A transcription regulatory element or sequence include, but is not limited to, a promoter sequence (e.g., the TATA box), an enhancer element, a signal sequence, or an array of transcription factor binding sites (response elements), that controls or regulates transcription of a gene operably linked to it.

A “variant” of a reference molecule refers to a molecule substantially similar in structure and biological activity to either the entire reference molecule, or to a fragment thereof. Thus, provided that two molecules possess a similar activity, they are considered variants as that term is used herein even if the composition or secondary, tertiary, or quaternary structure of one of the molecules is not identical to that found in the other, or if the sequence of amino acid residues is not identical.

A “vector” is a composition for facilitating introduction, replication and/or expression of a selected nucleic acid in a cell. Vectors include, e.g., plasmids, cosmids, viruses, YACs, bacteria, poly-lysine, etc. A “vector nucleic acid” is a nucleic acid molecule into which heterologous nucleic acid is optionally inserted which can then be introduced into an appropriate host cell. Vectors preferably have one or more origins of replication, and one or more sites into which the recombinant DNA can be inserted. Vectors often have convenient means by which cells with vectors can be selected from those without, e.g., they encode drug resistance genes. Common vectors include plasmids, viral genomes, and (primarily in yeast and bacteria) “artificial chromosomes.” “Expression vectors” are vectors that comprise elements that provide for or facilitate transcription of nucleic acids that are cloned into the vectors. Such elements can include, e.g., promoters and/or enhancers operably coupled to a nucleic acid of interest.

II. Identification of cDNAs Encoding Novel RTA-Modulatory Polypeptides

As used in the present invention, the consensus binding sites on a target gene that is regulated by RTA (i.e., an RTA responsive gene) are interchangeably termed “RTA recognition sequences,” “RTA response elements” or “RTA binding sites.” These sequences are found in many RTA responsive genes. Examples of RTA responsive genes include polyadenylated nuclear (PAN) RNA, kaposin (K12), ORF57, K-bZIP (K8, the ZEBRA homologue of KSHV), thymidine kinase, K5, ORF6 (single-stranded DNA binding protein), ORF59 (DNA polymerase-associated processivity factor), K14 (vOX-2), viral G protein-coupled receptor, and vIL-6. See, e.g., Chen et al., J. Virol. 74:8623-8634, 2000; Duan et al., Arch. Virol. 146:403-413, 2001; Haque et al., J. Virol. 74:2867-2875, 2000; Jeong et al., J. Virol. 75:1798-1807, 2001; Lukac et al., J. Virol. 75:6786-6799, 2001; Lukac, J. Virol. 73:9348-9361, 1999; Song et al., J. Virol. 75:3129-3140, 2001; Zhang et al., DNA Cell Biol. 17:735-742; 1998; and Deng et al., J Virol. 76:8252-64, 2002.

Other than trans-regulating expression of RTA responsive genes under its control, RTA also auto-regulates its own expression. For example, RTA binding sites have been identified upstream of the KSHV and EBV RTA genes. KSHV and EBV RTAs therefore form a feedback loop and auto-stimulate their own expression (Deng et al., J. Gen. Virol. 81:3043-3048, 2000; and Ragoczy et al., J. Virol. 75:5240-5251, 2001).

The present invention provides novel protein or polypeptide modulators that modulate RTA activities. Utilizing an expression vector which expresses a reporter gene under the control of an RTA response element, a number of polynucleotides were identified which up-regulate expression of the reporter gene when the expression vector and the polynucleotides were co-transfected into a host cell (see Example 1 below). An exemplary list of polynucleotides encoding such RTA-modulatory polypeptides is shown in Table 1. As shown in the Table, the novel RTA-modulatory polypeptides include diversified classes of proteins, including kinases and transcription factors.

As noted above, RTA also auto-regulates its own expression. Therefore, the up-regulation of reporter gene expression by the RTA modulatory polypeptides shown in Table 1 could be due to a direct inductive effect on the RTA response element in the expression construct and transcription of the reporter gene. Alternatively, the up-regulation could also be the result of enhanced expression or activity of endogenous RTA that in turn modulates expression of the reporter gene.

Thus, the RTA-modulatory polypeptides identified by the present inventors can operate with a number of mechanisms in modulating RTA activities. They can modulate upstream pathways leading to RTA activation (e.g., a kinase). Alternatively, the RTA-modulatory polypeptides could exert regulatory function on RTA expression or other biological activities of RTA. For example, they can stimulate RTA expression by, e.g., modulating events relating to transcription of the gene encoding RTA, modulating post-transcriptional processing of the RTA-encoding transcript, modulating the translation or post-translational modification of RTA, or modulating the stability or proteolysis of RTA. The RTA-modulatory polypeptides can also modulate other biological activities of RTA that are necessary for or involved in the transcription-regulating function of RTA (e.g., modulating the phosphorylation status of RTA or the DNA-binding activity of RTA).

TABLE 1
Polynucleotides encoding KSHV RTA-modulatory polypeptides

| | Accession | Description of the polynucleotide sequence and encoded | Fold of |
|---|---|---|---|
| | Number | polypeptide | modulation |
| 1 | BC000157 | Hypothetical protein LOC51058 | 5.6x |
| 2 | BC012841 | Homo sapiens, X-box binding protein 1, clone MGC: 8980 | 7.7x |
| | | IMAGE: 3856898, mRNA, complete cds | |
| 3 | BC005645 | Ets variant gene 1 | 42.7x |
| 4 | BC004695 | Similar to zinc finger protein 64 | 46.4x |
| 5 | BC003238 | Protein kinase, cAMP dependent, catalytic, alpha (Prkaca) | 164.6x |
| 6 | BC013572 | Similar to v-Ki-ras2 Kirsten rat sarcoma 2 viral oncogene | 148.1x |
| | | homolog | |
| 7 | BC016147 | Homo sapiens, clone MGC: 9485 IMAGE: 3921259, | 23.9x |
| | | mRNA, complete cds | |
| 8 | BC014296 | Similar to cyclin-dependent kinase inhibitor 1B (P27) | 13.8x |
| 9 | BC012694 | Similar to RAS-like protein expressed in many tissues | 39.2x |
| 10 | BC014290 | Mus musculus, clone MGC: 13959 IMAGE: 4038233, | 17.4x |
| | | mRNA, complete cds | |
| 11 | BC012696 | Similar to paired-like homeodomain transcription factor 1 | 21.0x |
| 12 | BC010588 | Mus musculus, E26 avian leukemia oncogene 1, 5′ | 18.0x |
| | | domain, clone MGC: 18571 IMAGE: 3676286, mRNA, | |
| | | complete cds | |
| 13 | BC011141 | Similar to growth arrest and DNA-damage-inducible 45 | 29.5x |
| | | alpha | |
| 14 | BC014727 | Mus musculus, clone MGC: 25480 IMAGE: 4487316, | 57.2x |
| | | mRNA, complete cds | |
| 15 | BC019729 | Mus musculus, clone MGC: 30688 IMAGE: 3969222, | 6.1x |
| | | mRNA, complete cds | |
| 16 | BC028994 | Mus musculus, clone MGC: 36628 IMAGE: 5355331, | 7.8x |
| | | mRNA, complete cds | |
| 17 | BC027372 | RIKEN cDNA 3100004P22 gene | 5.8x |
| 18 | BC034680 | RIKExN cDNA 8430401F14 gene | 15.1x |
| 19 | BC006499 | Homo sapiens Similar to v-Ha-ras Harvey rat sarcoma | 28x |
| | | viral oncogene homolog clone MGC: 2359 | |
| | | IMAGE: 2819996 mRNA complete cds | |
| 20 | NM_052854 | Homo sapiens old astrocyte specifically induced substance | 5.3x |
| | | (OASIS), mRNA | |
| 21 | XM_049037 | Homo sapiens trinucleotide repeat containing 9 (TNRC9), | 22.4x |
| | | mRNA | |
| 22 | NM_133330 | Homo sapiens Wolf-Hirschhorn syndrome candidate 1 | 9.8x |
| | | (WHSC1), transcript variant 1, mRNA | |
| 23 | AF208502 | Homo sapiens early B-cell transcription factor (EBF) | 6.1x |
| | | mRNA, partial cds | |
| 24 | NM_025021 | Homo sapiens mucoepidermoid carcinoma translocated 1 | 117.2x |
| | (BC028050) | (MECT1), mRNA (TORC1) | |
| 25 | BC053562 | Homo sapiens transducer of regulated cAMP response | 186.1x |
| | | element-binding protein (CREB) 2, mRNA (TORC2) | |
| 26 | AY360173 | Homo sapiens transducer of regulated CREB protein 3 mRNA | 340.6x |
| | | (TORC3) | |

III. Screening for Novel RTA Modulators

The RTA-modulatory polypeptides described above provide novel targets for screening for novel RTA modulators. Various biochemical and molecular biology techniques or assays well known in the art can be employed to practice the present invention. Such techniques are described in, e.g., Sambrook et al., Molecular Cloning: A Laboratory Manual, Cold Spring Harbor Press, N.Y., Second (1989) and Third (2000) Editions; and Ausubel et al., Current Protocols in Molecular Biology, John Wiley & Sons, Inc., New York (1987-1999).

A. Screening Scheme

Typically, test agents are first assayed for their ability to modulate a biological activity of an RTA-modulatory polypeptide (“the first assay step”) shown in Table 1. Modulating agents thus identified are then subject to further screening for ability to modulate an activity of RTA (e.g., its transcription-regulating activity), typically in the presence of the RTA-modulatory polypeptide (“the second testing step”). Depending on the RTA-modulatory polypeptide employed in the method, modulation of different biological activities of the RTA-modulatory polypeptide can be assayed in the first step. For example, a test agent can be assayed for binding to the RTA-modulatory polypeptide. The test agent can be assayed for activity to modulate the expression of the RTA-modulatory polypeptide, e.g., transcription or translation. The test agent can also be assayed for activities in modulating the cellular level or stability of the RTA-modulatory polypeptide, e.g., post-translational modification or proteolysis.

If the RTA-modulatory polypeptide has a known or well established biological or enzymatic function (e.g., kinase activity or DNA-binding activity), the biological activity monitored in the first screening step can be the specific biochemical or enzymatic activity of the RTA-modulatory polypeptide. In an exemplary embodiment, the RTA-modulatory polypeptide is a protein kinase (e.g., Prkaca encoded by a polynucleotide with accession number BC003238 in Table 1), and test agents are first screened for modulating the kinase's activity in phosphorylating a substrate. The substrate can be a polypeptide known to be phosphorylated by the kinase. The substrate may also be RTA or a fragment thereof Phosphorylation of RTA by the RTA-modulatory polypeptide can be examined by assays routinely practiced in the art.

Once test agents that modulate the RTA-modulatory polypeptides are identified, they are typically further tested for ability to modulate RTA activities. The test agent can be further tested for its ability to modulate expression level of RTA. Alternatively, the test agent can be further tested for its activity on modulating transcription-regulating function of RTA, e.g., binding to an RTA response element (e.g., the RTA response element in PAN promoter. Song et al., J. Virol. 75: 3129-40, 2001) or promoting expression of a gene under the control of an RTA response element (i.e., RTA responsive gene).

As noted above, the RTA-modulatory polypeptides identified by the present inventors can modulate expression level of RTA or transcription-regulating functions of RTA. If a test agent identified in the first screening step modulates expression level (e.g., by altering transcription activity) of the RTA-modulatory polypeptide, it would indirectly modulate RTA activities. For example, if the RTA-modulatory polypeptide (e.g., a kinase) modulates RTA activities by specifically phosphorylating RTA, a test agent which alters expression level of the RTA-modulatory kinase would indirectly also modulate RTA activities. Similarly, if the RTA-modulatory polypeptide modulates expression level of RTA, a test agent that modulates expression level of the RTA-modulatory polypeptide would indirectly alter expression level of RTA.

On the other hand, if a test agent modulates an activity other than expression level of the RTA-modulatory polypeptide, then the further testing step is needed to confirm that their modulatory effect on the RTA-modulatory polypeptide would indeed lead to modulation of RTA activities (e.g., expression level of RTA or transcription-regulating function of RTA). For example, a test agent, which modulates phosphorylation activity of an RTA-modulatory polypeptide, needs to be further tested in order to confirm that modulation of phosphorylation activity of the RTA-modulatory polypeptide can result in modulation of the transcription-regulating function or expression level of RTA.

In both the first assaying step and the second testing step, either an intact RTA-modulatory polypeptide and RTA, or their fragments, analogs, or functional derivatives can be used. The fragments that can be employed in these assays usually retain one or more of the biological functions of the RTA-modulatory polypeptide (e.g., kinase activity if the RTA-modulatory employed in the first assaying step is a kinase) or RTA (e.g., binding to an RTA response element). Fusion proteins containing such fragments or analogs can also be used for the screening of test agents. Functional derivatives of RTA-modulatory polypeptides and RTA usually have amino acid deletions and/or insertions and/or substitutions while maintaining one or more of the bioactivities and therefore can also be used in practicing the screening methods of the present invention.

A functional derivative can be prepared from a naturally occurring or recombinantly expressed RTA-modulatory polypeptide or RTA by proteolytic cleavage followed by conventional purification procedures known to those skilled in the art. Alternatively, the functional derivative can be produced by recombinant DNA technology by expressing only fragments of an RTA-modulatory polypeptide or RTA that retain one or more of their bioactivities.

A variety of well-known techniques can be used to identify test agents that modulate an RTA-modulatory polypeptide or RTA. Preferably, the test agents are screened with a cell based assay system. For example, in a typical cell based assay for screening RTA modulators (i.e., the second screening step), a construct comprising an RTA response element operably linked to a reporter gene is introduced into a host cell system (as exemplified in the Example below). The RTA-modulatory polypeptide can be expressed from a different vector that is also present in the host cell. In addition, RTA is usually also present in the assay, e.g., endogenously expressed by the host cell or expressed from another expression construct. The activity of the polypeptide encoded by the reporter gene (i.e., reporter polypeptide), e.g., an enzymatic activity, in the presence of a test agent can be determined and compared to the activity of the reporter polypeptide in the absence of the test agent. An increase or decrease in the activity identifies a modulator of RTA. The reporter gene can encode any detectable polypeptide (response or reporter polypeptide) known in the art, e.g., detectable by fluorescence or phosphorescence or by virtue of its possessing an enzymatic activity. The detectable response polypeptide can be, e.g., luciferase, alpha-glucuronidase, alpha-galactosidase, chloramphenicol acetyl transferase, green fluorescent protein, enhanced green fluorescent protein, and the human secreted alkaline phosphatase.

In addition to cell based assays described above, modulators of RTA can also be screened with non-cell based methods. For example, to identify agents that bind to the RTA-modulatory polypeptide, a number of non-cell based screening methods can be used. These methods include, e.g., mobility shift DNA-binding assays, methylation and uracil interference assays, DNase and hydroxy radical footprinting analysis, fluorescence polarization, and UV crosslinking or chemical cross-linkers. For a general overview, see, e.g., Ausubel et al., supra. One technique for isolating co-associating proteins, including nucleic acid and DNA/RNA binding proteins, includes use of UV crosslinking or chemical cross-linkers, including e.g., cleavable cross-linkers dithiobis (succinimidylpropionate) and 3,3′-dithiobis (sulfosuccinimidyl-propionate); see, e.g., McLaughlin (1996) Am. J. Hum. Genet. 59:561-569; Tang (1996) Biochemistry 35:8216-8225; Lingner (1996) Proc. Natl. Acad. Sci. USA 93:10712; Chodosh (1986) Mol. Cell. Biol 6:4723-4733.

B. Test Agents

Test agents that can be screened with methods of the present invention include polypeptides, beta-turn mimetics, polysaccharides, phospholipids, hormones, prostaglandins, steroids, aromatic compounds, heterocyclic compounds, benzodiazepines, oligomeric N-substituted glycines, oligocarbamates, polypeptides, saccharides, fatty acids, steroids, purines, pyrimidines, derivatives, structural analogs or combinations thereof. Some test agents are synthetic molecules, and others natural molecules.

Test agents are obtained from a wide variety of sources including libraries of synthetic or natural compounds. Combinatorial libraries can be produced for many types of compound that can be synthesized in a step-by-step fashion. Large combinatorial libraries of compounds can be constructed by the encoded synthetic libraries (ESL) method described in WO 95/12608, WO 93/06121, WO 94/08051, WO 95/35503 and WO 95/30642. Peptide libraries can also be generated by phage display methods (see, e.g., Devlin, WO 91/18980). Libraries of natural compounds in the form of bacterial, fungal, plant and animal extracts can be obtained from commercial sources or collected in the field. Known pharmacological agents can be subject to directed or random chemical modifications, such as acylation, alkylation, esterification, amidification to produce structural analogs.

Combinatorial libraries of peptides or other compounds can be fully randomized, with no sequence preferences or constants at any position. Alternatively, the library can be biased, i.e., some positions within the sequence are either held constant, or are selected from a limited number of possibilities. For example, in some cases, the nucleotides or amino acid residues are randomized within a defined class, for example, of hydrophobic amino acids, hydrophilic residues, sterically biased (either small or large) residues, towards the creation of cysteines, for cross-linking, prolines for SH-3 domains, serines, threonines, tyrosines or histidines for phosphorylation sites, or to purines.

The test agents can be naturally occurring proteins or their fragments. Such test agents can be obtained from a natural source, e.g., a cell or tissue lysate. Libraries of polypeptide agents can also be prepared, e.g., from a cDNA library commercially available or generated with routine methods. The test agents can also be peptides, e.g., peptides of from about 5 to about 30 amino acids, with from about 5 to about 20 amino acids being preferred, and from about 7 to about 15 being particularly preferred. The peptides can be digests of naturally occurring proteins, random peptides, or “biased” random peptides. In some methods, the test agents are polypeptides or proteins.

The test agents can also be nucleic acids. Nucleic acid test agents can be naturally occurring nucleic acids, random nucleic acids, or “biased” random nucleic acids. For example, digests of prokaryotic or eukaryotic genomes can be similarly used as described above for proteins.

In some preferred methods, the test agents are small molecules (e.g., molecules with a molecular weight of not more than about 1,000). Preferably, high throughput assays are adapted and used to screen for such small molecules. In some methods, combinatorial libraries of small molecule test agents as described above can be readily employed to screen for small molecule modulators of RTA. A number of assays are available for such screening, e.g., as described in Schultz (1998) Bioorg Med Chem Lett 8:2409-2414; Weller (1997) Mol Divers. 3:61-70; Fernandes (1998) Curr Opin Chem Biol 2:597-603; and Sittampalam (1997) Curr Opin Chem Biol 1:384-91.

Libraries of test agents to be screened with the claimed methods can also be generated based on structural studies of the RTA-modulatory polypeptides discussed above, RTA or its fragments. Such structural studies allow the identification of test agents that are more likely to bind to the RTA-modulatory polypeptides. The three-dimensional structures of the RTA-modulatory polypeptides or RTA can be studied in a number of ways, e.g., crystal structure and molecular modeling. Methods of studying protein structures using x-ray crystallography are well known in the literature. See Physical Bio-chemistry, Van Holde, K. E. (Prentice-Hall, New Jersey 1971), pp. 221-239, and Physical Chemistry with Applications to the Life Sciences, D. Eisenberg & D. C. Crothers (Benjamin Cummings, Menlo Park 1979). Computer modeling of RTA-modulatory polypeptides' structures provides another means for designing test agents for screening RTA modulators. Methods of molecular modeling have been described in the literature, e.g., U.S. Pat. No. 5,612,894 entitled “System and method for molecular modeling utilizing a sensitivity factor”, and U.S. Pat. No. 5,583,973 entitled “Molecular modeling method and system”. In addition, protein structures can also be determined by neutron diffraction and nuclear magnetic resonance (NMR). See, e.g., Physical Chemistry, 4th Ed. Moore, W. J. (Prentice-Hall, New Jersey 1972), and NMR of Proteins and Nucleic Acids, K. Wuthrich (Wiley-Interscience, New York 1986).

Modulators of the present invention also include antibodies that specifically bind to an RTA-modulatory polypeptide in Table 1. Such antibodies can be monoclonal or polyclonal. Such antibodies can be generated using methods well known in the art. For example, the production of non-human monoclonal antibodies, e.g., murine or rat, can be accomplished by, for example, immunizing the animal with an RTA-modulatory polypeptide in Table I or its fragment (See Harlow & Lane, Antibodies, A Laboratory Manual, Cold Spring Harbor Laboratory Press, Cold Spring Harbor N.Y.). Such an immunogen can be obtained from a natural source, by peptides synthesis or by recombinant expression.

Humanized forms of mouse antibodies can be generated by linking the CDR regions of non-human antibodies to human constant regions by recombinant DNA techniques. See Queen et al., Proc. Natl. Acad. Sci. USA 86, 10029-10033 (1989) and WO 90/07861. Human antibodies can be obtained using phage-display methods. See, e.g., Dower et al., WO 91/17271; McCafferty et al., WO 92/01047. In these methods, libraries of phage are produced in which members display different antibodies on their outer surfaces. Antibodies are usually displayed as Fv or Fab fragments. Phage displaying antibodies with a desired specificity are selected by affinity enrichment to an RTA-modulatory polypeptide in Table 1.

Human antibodies against an RTA-modulatory polypeptide in Table 1 can also be produced from non-human transgenic mammals having transgenes encoding at least a segment of the human immunoglobulin locus and an inactivated endogenous immunoglobulin locus. See, e.g., Lonberg et al., WO93/12227 (1993); Kucherlapati, WO 91/10741 (1991). Human antibodies can be selected by competitive binding experiments, or otherwise, to have the same epitope specificity as a particular mouse antibody. Such antibodies are particularly likely to share the useful functional properties of the mouse antibodies. Human polyclonal antibodies can also be provided in the form of serum from humans immunized with an immunogenic agent. Optionally, such polyclonal antibodies can be concentrated by affinity purification using an RTA-modulatory polypeptide in Table 1 or its fragment.

C. Screening Test Agents that Modulate RTA-Modulatory Polypeptides

A number of assay systems can be employed to screen test agents for modulators of an RTA-modulatory polypeptide. The screening can utilize an in vitro assay system or a cell-based assay system. In this screening step, test agents can be screened for binding to the RTA-modulatory polypeptide, altering expression level of the RTA-modulatory polypeptide, or modulating other biological activities of the RTA-modulatory polypeptide.

In some methods, binding of a test agent to an RTA-modulatory polypeptide is screened in the first screening step. Binding of test agents to an RTA-modulatory polypeptide can be assayed by a number of methods including e.g., labeled in vitro protein-protein binding assays, electrophoretic mobility shift assays, immunoassays for protein binding, functional assays (phosphorylation assays, etc.), and the like. See, e.g., U.S. Pat. Nos. 4,366,241; 4,376,110; 4,517,288; and 4,837,168; and also Bevan et al., Trends in Biotechnology 13:115-122, 1995; Ecker et al., Bio/Technology 13:351-360, 1995; and Hodgson, Bio/Technology 10:973-980, 1992. Agents that bind to the RTA-modulatory polypeptide can be identified by detecting a direct binding to the RTA-modulatory polypeptide, e.g., co-immunoprecipitation with the RTA-modulatory polypeptide by an antibody directed to the RTA-modulatory polypeptide. They can also be identified by detecting a signal that indicates that the agent binds to the RTA-modulatory polypeptide, e.g., fluorescence quenching or FRET.

Competition assays provide a suitable format for identifying test agents that specifically bind to an RTA-modulatory polypeptide. In such formats, test agents are screened in competition with a compound already known to bind to the RTA-modulatory polypeptide. The known binding compound can be a synthetic compound. It can also be an antibody, which specifically recognizes the RTA-modulatory polypeptide, e.g., a monoclonal antibody directed against the RTA-modulatory polypeptide. If the test agent inhibits binding of the compound known to bind the RTA-modulatory polypeptide, then the test agent also binds the RTA-modulatory polypeptide.

Numerous types of competitive binding assays are known, for example: solid phase direct or indirect radioimmunoassay (RIA), solid phase direct or indirect enzyme immunoassay (EIA), sandwich competition assay (see Stahli et al., Methods in Enzymology 9:242-253 (1983)); solid phase direct biotin-avidin EIA (see Kirkland et al., J. Immunol. 137:3614-3619 (1986)); solid phase direct labeled assay, solid phase direct labeled sandwich assay (see Harlow and Lane, “Antibodies, A Laboratory Manual,” Cold Spring Harbor Press (1988)); solid phase direct label RIA using ¹²⁵I label (see Morel et al., Mol. Immunol. 25(1):7-15 (1988)); solid phase direct biotin-avidin EIA (Cheung et al., Virology 176:546-552 (1990)); and direct labeled RIA (Moldenhauer et al., Scand. J. Immunol. 32:77-82 (1990)). Typically, such an assay involves the use of purified polypeptide bound to a solid surface or cells bearing either of these, an unlabelled test agent and a labeled reference compound. Competitive inhibition is measured by determining the amount of label bound to the solid surface or cells in the presence of the test agent. Usually the test agent is present in excess. Modulating agents identified by competition assay include agents binding to the same epitope as the reference compound and agents binding to an adjacent epitope sufficiently proximal to the epitope bound by the reference compound for steric hindrance to occur. Usually, when a competing agent is present in excess, it will inhibit specific binding of a reference compound to a common target polypeptide by at least 50 or 75%.

The screening assays can be either in insoluble or soluble formats. One example of the insoluble assays is to immobilize an RTA-modulatory polypeptide or its fragment onto a solid phase matrix. The solid phase matrix is then put in contact with test agents, for an interval sufficient to allow the test agents to bind. After washing away any unbound material from the solid phase matrix, the presence of the agent bound to the solid phase allows identification of the agent. The methods can further include the step of eluting the bound agent from the solid phase matrix, thereby isolating the agent. Alternatively, other than immobilizing the RTA-modulatory polypeptide, the test agents are bound to the solid matrix and the RTA-modulatory polypeptide molecule is then added.

Soluble assays include some of the combinatory libraries screening methods described above. Under the soluble assay formats, neither the test agents nor the RTA-modulatory polypeptide are bound to a solid support. Binding of an RTA-modulatory polypeptide or fragment thereof to a test agent can be determined by, e.g., changes in fluorescence of either the RTA-modulatory polypeptide or the test agents, or both. Fluorescence may be intrinsic or conferred by labeling either component with a fluorophor.

In some binding assays, either the RTA-modulatory polypeptide, the test agent, or a third molecule (e.g., an antibody against the RTA-modulatory polypeptide) can be provided as labeled entities, i.e., covalently attached or linked to a detectable label or group, or cross-linkable group, to facilitate identification, detection and quantification of the polypeptide in a given situation. These detectable groups can comprise a detectable polypeptide group, e.g., an assayable enzyme or antibody epitope. Alternatively, the detectable group can be selected from a variety of other detectable groups or labels, such as radiolabels (e.g., ¹²⁵I, ³²P, ³⁵S ) or a chemiluminescent or fluorescent group. Similarly, the detectable group can be a substrate, cofactor, inhibitor or affinity ligand.

Binding of a test agent to an RTA-modulatory polypeptide provides an indication that the agent can be a modulator of the RTA-modulatory polypeptide. It also suggests that the agent may modulate RTA activity (e.g., by binding to and modulating the RTA-modulatory polypeptide which in turn acts on RTA). Thus, a test agent that binds to an RTA-modulatory polypeptide can be further tested for the ability to modulate RTA (i.e., in the second testing step outlined above).

Alternatively, a test agent that binds to an RTA-modulatory polypeptide can be further examined to determine its activity on the RTA-modulatory polypeptide. The existence, nature, and extent of such activity can be tested by an activity assay. Such an activity assay can confirm that the test agent binding to the RTA-modulatory polypeptide indeed has a modulatory activity on the RTA-modulatory polypeptide. More often, such activity assays can be used independently to identify test agents that modulate activities of an RTA-modulatory polypeptide (i.e., without first assaying their ability to bind to the RTA-modulatory polypeptide). In general, such methods involve adding a test agent to a sample containing an RTA-modulatory polypeptide in the presence or absence of other molecules or reagents which are necessary to test a biological activity of the RTA-modulatory polypeptide (e.g., kinase activity if the RTA-modulatory polypeptide is a kinase), and determining an alteration in the biological activity of the RTA-modulatory polypeptide. In an exemplary embodiment, the RTA-modulatory polypeptide is a kinase (e.g., Prkaca encoded by BC003238), and the test agent is examined for ability to modulate the kinase activity of the RTA-modulatory polypeptide. Methods for monitoring kinase activity are well known in the art, e.g., as described in Sambrook et al. and Ausubel et al., supra.

In addition to assays for screening agents that modulate an enzymatic or other biological activities of an RTA-modulatory polypeptide, the activity assays also encompass in vitro screening and in vivo screening for alterations in expression level of the RTA-modulatory polypeptide.

D. Screening for Agents that Modulate RTA Activities

Once a modulating agent has been identified to bind to an RTA-modulatory polypeptide and/or to modulate a biological activity (including expression level) of the RTA-modulatory polypeptide, it can be further tested for ability to modulate RTA activities. Modulation of RTA activities by the modulating agent is typically tested in the presence of the RTA-modulatory polypeptide. When a cell-based screening system is employed, the RTA-modulatory polypeptide can be expressed from an expression vector that has been introduced into a host cell. RTA can be expressed from a second expression vector. Alternatively, RTA can be endogenously expressed by the host cell in the screening system (e.g., KS-1 cell as discussed in the Example below).

Unless otherwise specified, modulation of RTA includes modulation of any of the biological activities of RTA in regulating viral transcription and infection of the host cell. Thus, the term “RTA activity” or “biological activity of RTA” encompasses transcription-regulating activities of RTA and activities affecting the expression level of RTA (e.g., transcription activities). Any of these activities can be tested in the presence of a modulating agent that has been identified to bind to and/or modulate an RTA-modulatory polypeptide. For example, activities of RTA to be monitored in this screening step include activities relating to the expression level of RTA (e.g., transcription), enzymatic or non-enzymatic modification of RTA protein, and biochemical activities of expressed RTA proteins (binding to an RTA response element or regulating expression of a gene under the control of a RTA response element).

Modulation of expression level or other activities of RTA can be determined in a non-cell based assay system or cell-based assays, similar to the first screening step for identifying modulators of RTA-modulatory polypeptides. Using eukaryotic in vitro transcription systems, effects of test agents on RTA level or activities can be tested by directly measuring expression level or transcription-regulating activity of RTA in the presence of the test agents. Because the test agent is likely to exert its modulatory effect on RTA by modulating an RTA-modulatory polypeptide, the RTA-modulatory polypeptide is typically also-present in the assay system.

With cell-based assays, vectors expressing a reporter gene or other linked polynucleotides under the control of an RTA response element (e.g., a promoter or an enhancer sequence) are introduced into appropriate host cells. Modulation of RTA activities are typically examined by measuring expression of the reporter gene or other linked polynucleotides. An altered activity of the reporter gene (e.g., its expression level) in the presence of a test agent would indicate that the test agent is a modulator of RTA activity. Activities of RTA can be examined with assays routinely practiced in the art. For example, expression of a reporter gene under the control of a RTA response element can be measured using methods as described in, e.g., Sambrook et al., supra; and Ausubel et al., supra. Alternatively, methods described in the Example below can be used to monitor effects of modulating agents on transcription-regulating activity of RTA.

Similar to the first screening step, modulation of RTA expression level or its transcription-regulating activities can be examined in a cell-based system by transient or stable transfection of an expression vector into cultured cell lines. For monitoring expression level of RTA, assay vectors bearing a RTA transcription regulatory sequence (promoter or enhancer sequences) operably linked to a reporter gene can be used. To assess effects of modulating agents on transcription-regulating activity of RTA, the assay vectors harbor an RTA response element that is operably linked to a reporter gene. In addition to the RTA response element, the expression vectors can contain additional transcription regulatory sequences such as a promoter (e.g., a PAN promoter as discussed in the Example below).

Constructs containing an RTA transcription regulatory element or RTA response element that is operably linked to a reporter gene can be prepared using only routinely practiced techniques and methods of molecular biology (see, e.g., Sambrook et al. and Ausubel et al., supra). One example of such constructs is pLUC/-69 as described in Song et al., J. Virol. 75: 3129-40, 2001. The assay vectors can be transfected into any mammalian cell line (e.g., KS-I cell line as described in the Example) to assay expression of the reporter gene. General methods of cell culture, transfection, and reporter gene assay have been described in the art, e.g., Ausubel, supra; and Transfection Guide, Promega Corporation, Madison, Wis. (1998). Other than the KS-1 cell line, other transfectable mammalian cell lines may also be used, e.g., HEK 293, MCF-7, and HepG2 cell lines. If the host cells do not express RTA endogenously, a separate vector expressing the RTA protein can be co-transfected into the host cells.

When inserted into the appropriate host cell, the RTA transcription regulatory element or RTA response element in the expression vector induces transcription of the reporter gene by host RNA polymerases. Reporter genes typically encode polypeptides with an easily assayed enzymatic activity that is naturally absent from the host cell. Typical reporter polypeptides for eukaryotic promoters include, chloramphenicol acetyltransferase (CAT), firefly or Renilla luciferase, beta-galactosidase, beta-glucuronidase, alkaline phosphatase, and green fluorescent protein (GFP).

Transcription driven by an RTA transcription regulatory element or RTA response element may also be detected by directly measuring the amount of RNA transcribed from the reporter gene. In these embodiments, the reporter gene may be any transcribable nucleic acid of known sequence that is not otherwise expressed by the host cell. RNA expressed from constructs containing an RTA transcription regulatory element or RTA response element may be analyzed by techniques known in the art, e.g., reverse transcription and amplification of mRNA, isolation of total RNA or poly A⁺ RNA, northern blotting, dot blotting, in situ hybridization, RNase protection, primer extension, high density polynucleotide array technology and the like. These techniques are all well known and routinely practiced in the art.

Other than monitoring RTA transcription-regulating activity or its expression level, effects of modulating agents on RTA function can also be screened for ability to modulate viral replication. RTA plays an important role in activating viral lytic replication. Effects of the modulating agents on such activity of RTA can be tested with KSHV virus in PEL cell lines, e.g., as described in Lukac et al., J. Virol. 74: 9348-61, 1999; and Sun et al., Proc. Natl. Acad. Sci. USA 95: 10866-71, 1998. In some other methods, the modulating agents identified in the first screen step can also be screened for effects on expression of RTA responsive genes. Expression of a number of genes are known to be regulated by the RTA, including the gene encoding RTA itself and several downstream genes such as polyadenylated nuclear (PAN) RNA, kaposin (K12), ORF57, K-bZIP (K8, the ZEBRA homologue of KSHV), thymidine kinase, K5, ORF6 (single-stranded DNA binding protein), ORF59 (DNA polymerase-associated processivity factor), K14 (vOX-2), viral G protein-coupled receptor, and vIL-6. Modulating agents that alter expression level of a RTA responsive gene through interacting with RTA can be confirmed as RTA modulators. Such screening can be performed as described in the art, e.g., in Lukac et al., J. Virol. 74: 9348-61, 1999; and Sun et al., Proc. Natl. Acad. Sci. USA 95: 10866-71, 1998.

IV. Therapeutic Applications

The present invention provides compositions and methods for treating infections of gammaherpesvirus in various subjects including human. There are a number of diseases and conditions that are mediated by or are associated with gammaherpesvirus. As noted above, Kaposi's sarcoma herpesvirus (KSHV or HHV-8) is a cofactor in various forms of Kaposi's sarcoma and several other diseases. Epstein-Barr virus (human herpesvirus 4) is associated with human cancers. All these diseases and conditions that are associated with infections of gammaherpesviruses can be treated with the novel RTA modulators of the present invention which inhibit RTA biological activities. Modulation of RTA activity or expression levels is also useful for preventing or modulating the development of such diseases or disorders in a subject (e.g., human or non-human mammals) of being, or known to be, prone to infections of gammaherpesviruses.

Modulators that inhibit RTA activity can be administered directly to a subject that is infected by a gammaherpesvirus (e.g., KSHV). Such modulators include small molecule compounds identified in accordance with the present invention, as well as an antibody or an siRNA against a polypeptide modulator of RTA (e.g., as shown in Table 1). The modulators can be administered alone or as the active ingredient of a pharmaceutical composition. Administration can be by any of the routes which are well known to those of skill in the art and which are normally used for introducing a modulating compound into ultimate contact with the tissue to be treated.

The RTA-inhibiting modulators of the present invention can be administered to a subject at therapeutically effective doses to prevent, treat, or control diseases or conditions associated with infections of gammaherpesvirus, e.g., KSHV. The compounds are administered to a subject in an amount sufficient to elicit an effective protective or therapeutic response in the subject. An effective protective or therapeutic response is a response that at least partially arrests or slows the symptoms or complications of the disease. An amount adequate to accomplish this is defined as “therapeutically effective dose.” The optimal dose level for any subject will depend on a variety of factors including the efficacy of the specific modulator employed, the age, body weight, physical activity, and diet of the subject, and on a possible combination with other drug. The size of the dose also will be determined by the existence, nature, and extent of any adverse side-effects that accompany the administration of a particular compound or vector in a particular subject.

In determining the effective amount of the modulator to be administered, a physician may evaluate circulating plasma levels of the modulator, modulator toxicity, and the production of anti-modulator antibodies. In general, the dose equivalent of a modulator is from about 1 ng/kg to 10 mg/kg for a typical subject.

For administration, modulators of the present invention can be administered at a rate determined by the LD-50 of the modulator, and the side-effects of the modulator at various concentrations, as applied to the mass and overall health of the subject. Administration can be accomplished via single or divided doses.

The modulators of the invention may be used alone or in conjunction with other agents that are known to be beneficial in treating or preventing human diseases that are mediated by gammaherpesviruses, e.g., KSHV or EBV infections. The modulators of the invention and another agent may be co-administered, either in concomitant therapy or in a fixed combination, or they may be administered at separate times. There are many known antiviral agents which can be employed in the present invention. Examples include interferons, nucleoside analogues, ribavirin, amantadine, and pyrophosphate analogues of phosphonoacetic acid (foscarnet) and the like (Gorbach et al., Infectious Disease, Ch.35, p. 289, W. B. Saunders (Ed.), Philadelphia, Pa., 1992). Many antiherpesvirus nucleoside analogs can also be used (e.g., as described in Balzarini et al., Mol. Pharm. 37,402-7, 1990). These nucleoside analogs act through inhibition of viral DNA replication, especially through inhibition of viral DNA polymerase. A number of specific compounds which are useful as anti-herpesviral agents are also disclosed in U.S. Pat. No. 6,500,663.

The pharmaceutical compositions of the invention may comprise a pharmaceutically acceptable carrier. Pharmaceutically acceptable carriers are determined in part by the particular composition being administered, as well as by the particular method used to administer the composition. There are a wide variety of suitable formulations of pharmaceutical compositions of the present invention (see, e.g., Remington: The Science and Practice of Pharmacy, Mack Publishing Co., 20^{th} ed., 2000).

Formulations suitable for administration include aqueous and non-aqueous solutions, isotonic sterile solutions, which can contain antioxidants, buffers, bacteriostats, and solutes that render the formulation isotonic, and aqueous and non-aqueous sterile suspensions that can include suspending agents, solubilizers, thickening agents, stabilizers, and preservatives. In the practice of this invention, compositions can be administered, for example, orally, nasally, topically, intravenously, intraperitoneally, intrathecally or into the eye (e.g., by eye drop or injection). The formulations of compounds can be presented in unit-dose or multi-dose sealed containers, such as ampoules and vials. Solutions and suspensions can be prepared from sterile powders, granules, and tablets of the kind previously described. The modulators can also be administered as part of a prepared food or drug.

### EXAMPLES

The following examples are provided to illustrate, but not to limit the present invention.

### Example 1

### Identification of cDNAs Encoding Modulators of KSHV/HHV-8 RTA

This Example describes the identification of various RTA modulatory polypeptides that regulate expression of a reporter gene under the control of an RTA-responsive element.

Two arrayed and annotated cDNA libraries (GNF Mammalian Gene Collection and Origene Collection) inserted into mammalian expression vectors were interrogated for modulators of HHV-8/KSHV RTA expression and activity. These libraries, consisting of approximately 11,000 (MGC) and 15,000 (Origene) full length mammalian cDNAs were spotted in 384-well plates such that each well contained an individual cDNA of known identity. In a semi-automated process, cDNAs were incubated with a non-liposomal transfection reagent (Fugene6, Roche Applied Science, Indianapolis, Ind.) and pLUC/-69, a construct containing a minimal PAN promoter driving the firefly luciferase gene. pLUC/-69 has been described in Song et al. (Journal of Virology 75: 3129-3140), and was used as a barometer for RTA function. It contains a very strong RTA response element from the PAN promoter. This element is present in a subset of genes regulated by RTA (Song et al., Journal of Virology 76:5000-5013). In the context of the HHV8 lytic cycle, the PAN promoter (from which the pLUC/-69 construct was derived) drives the expression of an abundant noncoding polyadenylated nuclear RNA that comprises ˜80% of the total poly(A)-selected transcripts in infected cells, thus providing a robust screen read out.

KS-1 cells were then introduced into each well to complete the transfection process. KS-1 cells are derived from a primary effusion lymphoma and harbor latent HHV-8 (described in Said et al. Blood 87:4937). After 2 (Origene) or 3 (MGC) days of incubation at 37 C and 5% CO₂, an equal volume of Bright-Glo reagent (Promega, Madison, Wis.) was added to each well and relative luminescence was determined using an Acquest (LJL Biosystems, Sunnyvale, Calif.) plate reader.

After executing the assay in duplicate, plate data were normalized to a median value and compared across the respective libraries (11,000 wells for the MGC collection, and 15,000 wells for the Origene cDNA collection). Approximately 67 cDNAs with mean activity values more than 3 fold greater than the whole experimental mean were selected from the library, and subsequently amplified and isolated using commercially available DNA isolation reagents (Qiagen, Germany). These samples were reconfirmed utilizing the methods outlined above. cDNAs possessing confirmed modulatory activity on the pLUC/-69 reporter construct are listed in Table 1 above.

### Example 2

### Characterization of cDNAs Encoding Modulators of KSHV/HHV-8 RTA

This Example describes additional studies demonstrating that several cDNA hits identified in the primary screen are indeed critical mediators of HHV-8 reactivation. First, using an RTA promoter-luciferase reporter construct in 293T cells, it was shown that RTA expression is directly activated by Prkaca (BC003238). In the primary screen with KS-1 cells described in Example 1, activated RTA expression presumably leads to the upregulated expression from the PAN promoter (PAN promoter activation was the basis for its identification in the primary screen). It was also shown that this activation of the PAN promoter in KS-1 cells by Prkaca is RTA-mediated as it is dependent on an intact RTA binding site in the promoter. Prkaca further appears to be able to synergistically activate the PAN promoter in combination with RTA, suggesting that it acts during two distinct stages in reactivation—one in the direct activation of RTA expression, and secondly as a potent co-activator at RTA responsive promoters.

In addition, reactivation (as measured by PAN promoter activation) by Prkaca also appears to be CREB dependent, as the co-introduction of a dominant negative CREB (A-CREB) into KS-1 cells abrogates this response. CREB (cAMP-response element binding protein) is a ubiquitous transcription factor which binds to the cAMP response element (CRE) and stimulates transcription after phosphorylation on Ser(133) by PKA (Prkaca is the catalytic subunit of PKA). A-CREB is described in Conkright et al. (Molecular Cell vol. 12:413-423, 2003).

Further, another primary screen hit, NM_{—}025021 (also known as TORC1), was found to be sufficient to induce activation of reporter driven by the PAN promoter (pPAN-69Luc) in KS-1 cells. TORC1 was first described in Conkright et al. as a CREB co-activator. A model of the relationship between TORC1 and CREB may be found in Conkright et al. This activity was found to be dependent on the presence of an RTA binding site in the PAN promoter. Reactivation by TORC1, as with Prkaca, was also found to require CREB function, as the introduction of a dominant negative mutant form of CREB abrogated reactivation. TORC1 was also found to directly affect RTA promoter activity, and to act synergistically with RTA in activating an RTA-responsive promoter.

These studies indicate that CREB, Prkaca, and TORC1 are critical mediators of HHV-8 reactivation. Moreover, the convergence at CREB suggest that CREB phosphorylation by PKA (Prkaca) could present a point of pharmacological intervention by which HHV-8 reactivation can be countered.

It is understood that the examples and embodiments described herein are for illustrative purposes only and that various modifications or changes in light thereof will be suggested to persons skilled in the art and are to be included within the spirit and purview of this application and scope of the appended claims. Although any methods and materials similar or equivalent to those described herein can be used in the practice or testing of the present invention, the preferred methods and materials are described.

All publications, GenBank sequences, ATCC deposits, patents and patent applications cited herein are hereby expressly incorporated by reference in their entirety and for all purposes as if each is individually so denoted.

1. A method for identifying an agent that modulates the replication and transcription activator (RTA) of a gammaherpesvirus, the method comprising:
(a) assaying a biological activity of an RTA-modulatory polypeptide encoded by a polynucleotide selected from the members listed in Table 1, or a fragment of said polypeptide, in the presence of a test agent to identify one or more modulating agents that modulate the biological activity of the polypeptide; and
(b) testing one or more of the modulating agents for ability to modulate a biological activity of the RTA.

2. The method of claim 1, wherein the gammaherpesvirus is human Kaposi's sarcoma virus (human herpesvirus 8) or Epstein-Barr virus.

3. The method of claim 1, wherein the modulating agents inhibit the biological activity of the polypeptide.

4. The method of claim 1, wherein (b) comprises testing the modulating agents for ability to modulate RTA in regulating expression of an RTA responsive gene.

5. The method of claim 1, wherein (b) comprises testing the modulating agents for ability to modulate RTA in inducing expression of a second polynucleotide that is operably linked to an RTA response element.

6. The method of claim 5, wherein the second polynucleotide encodes a reporter polypeptide.

7. The method of claim 5, wherein the testing for ability to modulate the biological activity of the RTA comprises:
providing a cell or cell lysate that comprises the second polynucleotide that is operably linked to the RTA response element;
contacting the cell or cell lysate with a test agent; and
detecting an increase or decrease in expression of the second polynucleotide in the presence of the test agent compared to expression of the second polynucleotide in the absence of the test agent.

8. The method of claim 1, wherein (b) comprises testing the modulating agents for ability to modulate expression level of the RTA.

9. The method of claim 1, wherein the RTA-modulatory polypeptide is a kinase.

10. The method of claim 1, wherein the polynucleotide has an accession number of BC003238 or NM_{—}025021.

11. The method of claim 1, wherein the assaying of the biological activity of the RTA-modulatory polypeptide occurs in a cell.

12. The method of claim 11, wherein the RTA-modulatory polypeptide is expressed from said polynucleotide that has been introduced into the cell.

13. A method for identifying an agent that modulates expression of an RTA responsive gene, the method comprising:
(a) contacting a test agent with an RTA-modulatory polypeptide encoded by a polynucleotide selected from the members listed in Table 1;
(b) detecting a change in an activity of said RTA-modulatory polypeptide relative to the activity in the absence of the test agent; and
(c) detecting a change of expression level of the RTA responsive gene in the presence of the test agent identified in (b) relative to expression level of the RTA responsive gene in the absence of the test agent; thereby identifying the test agent as a modulator of expression of the RTA responsive gene.

14. The method of claim 13, wherein (a) and (b) are performed in a cell.

15. A method for inhibiting replication of a gammaherpesvirus in a subject, comprising administering to the subject a pharmaceutical composition comprising an effective amount of a modulator of the replication and transcription activator (RTA) of the gammaherpesvirus, thereby inhibiting replication of the gammaherpesvirus, wherein the RTA modulator is identified by:
(a) assaying a biological activity of an RTA-modulatory polypeptide encoded by a polynucleotide selected from the members listed in Table 1, or a fragment of said polypeptide, in the presence of a test agent to identify one or more modulating agents that inhibit the biological activity of the polypeptide; and
(b) testing one or more of the modulating agents for ability to inhibit transcription-regulating activity of the RTA.

16. The method of claim 15, wherein the gammaherpesvirus is human Kaposi's sarcoma virus (human herpesvirus 8) or Epstein-Barr virus.

17. The method of claim 15, wherein the subject is a human.

18. The method of claim 15, wherein the RTA-modulatory polypeptide is a kinase.

19. The method of claim 15, wherein the modulating agents are tested for ability to inhibit the RTA activity in regulating expression of an RTA responsive gene.

20. The method of claim 15, wherein the modulating agents are tested for ability to inhibiting the RTA activity in inducing expression of a second polynucleotide that is operably linked to an RTA response element.

21. The method of claim 20, wherein the second polynucleotide encodes a reporter polypeptide.

22. The method of claim 20, wherein the modulating agents are tested for ability to inhibit the RTA activity in a host cell.

23. The method of claim 20, wherein the host cell expresses the RTA, and the second polynucleotide has been introduced into the cell.

24. The method of claim 20, wherein the host cell is KS-1 cell.
